(19) United States
(12) Patent Application Publication
(10) Pub. No.: US 2012/0128787 A1
(43) Pub. Date: May 24, 2012

(54) Homeopathic Composition Comprising Hypericum Perforatum Extract And Essential Oils For The Treatment Of Neuropathic Pain

(75) Inventor: Alexander Mclellan, Ketch Harbour (CA)

(73) Assignee: ORIGIN BIOMED INC., Halifax, NS (CA)

(21) Appl. No.: 13/130,024

(22) PCT Filed: Nov. 18, 2009
(86) PCT No.: PCT/CA2009/001630

§ 371 Date: Sep. 28, 2011

Related U.S. Application Data
(60) Provisional application No. 61/115,778, filed on Nov. 18, 2008.

(51) Int. Cl. A61K 33/42; A61K 36/38; A61K 36/61; A61P 31/00; A61K 36/899; A61K 36/77; A61K 36/714; A61P 29/00; A61K 36/00; A61P 35/00

(52) U.S. Cl. 424/601; 424/725; 424/730; 424/742

(57) ABSTRACT

The present invention provides a method of treating neuropathic pain by administering a topical homeopathic composition to a mammalian subject. The homeopathic formulation contains homeopathic active ingredients comprising Hypericum perforatum in a base of essential oils which facilitate delivery of the homeopathic ingredient through the skin.

### CROSS-REFERENCE TO RELATED APPLICATION

This application claims the benefit and priority to U.S. provisional patent application No. 61/115,778, filed Nov. 18, 2008, which is incorporated herein in its entirety as though set forth explicitly herein.

### FIELD OF THE INVENTION

The present invention pertains to the field of homeopathic compositions and methods of use thereof to treat neuropathic pain. More particularly, the present invention pertains to the field of homeopathic compositions comprising a base for improved penetration of the homeopathic agent through the skin.

### BACKGROUND

Neuropathic pain is pain caused by various types of nerve damage, including but not limited to diabetic peripheral neuropathy, herpes zoster, post herpetic neuralgia, trigeminal neuralgia, complex regional pain syndrome, reflex sympathetic dystrophy, phantom limb syndrome, neuropathic pain due to chronic disease (multiple sclerosis, HIV, etc), neuropathic pain due to trauma (causalgia), neuropathic pain due to impingement (i.e., sciatica, carpal tunnel, etc.), neuropathic pain due to drug exposure or toxic chemical exposure, neuropathic pain due to infection or post infection, neuropathic pain due to impaired organ function, neuropathic pain due to vascular disease, neuropathic pain due to metabolic disease, neuropathic pain due to cancer or cancer treatment, neuropathic pain due to autoimmune disease, neuropathic pain due to fibromylagia, and neuropathic pain with no know cause (idiopathic) as well as treating any pain is that is characterized by burning sensations and/or shooting pain and/or numbness and/or tingling and/or allodynia.

This type of pain is typically associated with one or more of he characteristics below:
Allodynia: pain due to a stimulus that normally does not cause pain, for example as the light touch from air passing over skin.
Hyperpathia: A painful syndrome characterized by increased reaction to a stimulus, especially a repetitive stimulus, as well an increased threshold.
Hyperesthesia: An increased sensitivity to normal stimulation excluding the special senses.
Hyperalgesia: An increased response to a stimulus that is normally painful.
Dysesthesia: An unpleasant abnormal sensation, whether spontaneous or evoked.
Paresthesia: An abnormal sensation, whether spontaneous or evoked.
Deafferentation Pain: Pain due to loss of sensory input into the central nervous system, as occurs with lesions of the peripheral nerves or due to pathology of the central nervous system.
Anesthesia Delorosa: Pain in an area or region that is anesthetic (absence of all sensations).

It is well known that nociceptive pain and neuropathic pain are caused by different mechanisms, and therefore respond to different treatment modalities. Nociceptive pain is mediated by receptors which are located in skin, bone, connective tissue, muscle and viscera. These receptors typically respond to noxious chemical, thermal and mechanical stimuli producing pain that is typically described as sharp, aching, throbbing, or gnawing. In contrast, neuropathic pain is produced by damage to, or pathological changes in, the peripheral or central nervous systems, typically producing pain that is described as “burning”, “electric”, “tingling”, and “shooting” in nature. Finally, nociceptive pain usually responds to opioids and non-steroidal anti-inflammatories (NSAIDS), whereas success treating neuropathic pain with these approaches has been limited. Conversely, agents employed to treat neuropathic pain, such as gabapentin, have little or no effect on nociceptive pain.

Current conventional pharmacologic strategies for treating neuropathic pain follow a number of different approaches as outlined below.

Antiarrhythmics: Certain antiarrhythmics have sodium-blocking activity. Low-dose IV lidocaine is sometimes used for temporary pain relief from peripheral nervous system injuries, including diabetic neuropathy and postherpetic neuralgia. However, IV lidocaine therapy requires constant monitoring of the patient's ECG and blood pressure to decrease the risk for seizures and arrhythmias.(1)

Antidepressants: Both tricyclic antidepressants and serotonin reuptake inhibitors are have been used to treat neuropathic pain. Numerous clinical trials demonstrate the safety and efficacy of TCAs when used to treat either diabetic neuropathy or postherpetic neuralgia, yet response rates have been low at approximately 33%. Amitriptyline was the first tricyclic used to treat neuropathy, and it is still widely prescribed. Amitriptyline has a high incidence of anticholinergic side effects, including delirium in elderly patients. TCAs also have proarrhythmic effects which limit their use in populations with abnormal EKG. Serotonin specific reuptake inhibitors (SSRIs) have demonstrated less consistent effects on neuropathic pain, relieving neuropathic pain in only one of seven patients. Serotonin noradrenaline reuptake inhibitors have fared slightly better with a response rate of one in every 4-5 neuropathic pain sufferers. (2)

Anticonvulsants: Carbamazepine, phenytoin, gabapentin and lamotrigine have all been used to treat neuropathic pain. Inhibition of sodium channel blocking activity by agents such as carbamazepine, phenytoin, and lamotrigine is the proposed mechanism. Studies have shown the anticonvulsant gabapentin to be effective in painful diabetic neuropathy, mixed neuropathies, and postherpetic neuralgia. The most common adverse effects of anticonvulsants in general are sedation and cerebellar symptoms (nystagmus, tremor and incoordination). The most common side effects associated with gabapentin are asthenia, headache, dizziness and somnolence, and in some cases polyneuropathy. Lamotrigine was no better than placebo when used to treat neuropathic pain other than trigeminal neuralgia. (3)

NSAIDS: NSAIDS are not generally recommended first-line agents for treating neuropathic pain. Relief of neuropathic pain with nonsteroidal anti-inflammatory drugs (NSAIDs) is variable. (4)

Opioids: Treatment of neuropathic pain has with opioids has been controversial. Opioids were thought to be ineffective for treating neuropathic pain, but may be somewhat effective for patients who have failed other modalities. Short-term studies provide only equivocal evidence regarding the efficacy of opioids in reducing the intensity of neuropathic pain, while intermediate-term studies demonstrate significant efficacy of opioids over placebo. Reported adverse events of opioids are common and long-term efficacy, safety (including addiction potential), and effects on quality of life need to be further evaluated. Overall, neuropathic pain may be less responsive to opioids than other types of pain. (5)

Overall, the efficacy of these pharmacological treatments is often limited by side effects at the doses required for analgesia, as well as in some cases long delays before the onset of analgesia, a substantial rate of non responsiveness to therapy, and a potential for addiction.

Topical Agents: Topical agents offer the advantage of local relief without systemic toxicity. A new and novel non-toxic topical preparation to treat neuropathic pain is therefore of great interest and has the potential to benefit a wide range of chronic pain sufferers. There is a need for a safe, OTC topical pain relieving product that relieves most neuropathic pain within a few minutes, providing relief that lasts up to several hours (for uninterrupted sleep and work), and without unpleasant side effects such as counterirritation, redness, itching, stinging, cooling, sensitization, staining, burning, anesthesia, etc. Ideally such product would also not interfere or interact with oral prescription pain medications.

Homeopathy is widely accepted as a useful therapeutic throughout Europe, the British Commonwealth countries and India, and has been demonstrated to have characteristic and reproducible effects. A critical review of more than 100 controlled and/or clinical studies of homeopathy determined that patients received positive healing benefits from homeopathy beyond the placebo effect (Kleijnen, J. et al. 1991 Brit. Med. J. 302:316-323; Linde, K., Clausius, N., Ramirez, G., Melchart, D., Eitel, F., Hedges, L. V., Jonas, W. B., 1997, Lancet, 350:834-843; Reilly, D., et al, 1994, Lancet, 344:1601-1608). One of the basic tenets of homeopathic medicine is that a cure for a disease can be evoked by using a high dilution medicine that resembles but is different from the cause of the disease.

After a base preparation is made, either by an extract or maceration of an herbal compound or the dissolving of a selected compound in a solvent, a series of dilutions are prepared from the initial batch, called the “mother tincture”. Homeopathic drugs are diluted according to either the decimal “X” or centesimal “C” scales. For a “3×” preparation, the mother tincture is diluted with nine parts of the desired diluent, in either liquid or powder form. The resultant mixture is then diluted a second time, in a ratio of one part mixture to ten parts solvent and the resulting mixture is diluted a third time in a ration of one to ten. Therefore, the 3× drug is actually at 10⁻³ potency of the mother tincture. Similarly, a 6× dilution would be at 10⁻⁶ potency of the original solution. In the “C scale” each dilution is done with ninety-nine parts diluent to the original mixture. Therefore, a “3C” solution is at 10⁻⁶ potency of the original mixture and thus corresponds to a 6× potency. These scales are recognized by the Homeopathic Pharmacopoeia of the United States (H.P.U.S.).

U.S. Pat. No. 7,229,648 entitled “Homeopathic formulations useful for treating pain and/or inflammation”, teaches the use of homeopathic active drug ingredients for the treatment of pain. However, the homeopathic ingredients chosen and claimed focus on the treatment of nociceptive pain rather than neuropathic pain conditions. Current medical science recognizes a clear distinction between nociceptive and neuropathic pain. Further, Dreyer teaches the use of water based homeopathies only, which are not suited for topical delivery of the active drug to the nerves that are the source of the pain signals.

A number of references cite the use of essential oils as enhancers of skin penetration and therefore useful carriers for the absorption of pharmaceutical active ingredients. For example, Abdullah teaches that various essential oils enhance the absorption of 5-fluorouracil, a commonly used anti-neoplastic agent, through rat skin (6). Since Abdullah teaches the use of a hydrophilic base, however, absorption of the active ingredients across the cell membranes was limited by the solubility of the essential oils in the hydrophilic base. Further, Abdullah and similar references make no mention of the use of homeopathic active ingredients.

In United States Patent Application #20060275509, Wegener teaches the use of essential oils to achieve improved absorption of pharmaceutical agents and specifically polyphenols for the prevention of viral eruptions of the skin. In United States Patent Application #20030161867 Lu teaches the use of essential oil components such as terpenes, terpenoids, fatty alcohols and derivatives thereof as skin permeation enhancers for the delivery of a selective COX-2 inhibitory drug to a site of pain and/or inflammation, by application to an overlying or adjacent position to the site of pain/ and or inflammation. U.S. Pat. No. 6,132,760 describes a topical transdermal patch for the delivery of testosterone containing terpenes as a delivery enhancing adjuvant. These documents relate to the use of specific non-homeopathic active ingredients and do not mention the use of homeopathic agents as active ingredients. Furthermore, these documents do not include any teaching relevant to the treatment of neuropathic pain.

In U.S. Pat. No. 6,579,543, McClung teaches the use of a topical composition for the relief of pain containing a variety of compounds, including an analgesic, an antioxidant, an anti-neuralgic, an anti-inflammatory, an antidepressant and a blood circulation enhancer. The active analgesic is chosen from a laundry list of substances not prepared in traditional homeopathic method but crude herbal forms. Furthermore, McClung does not reference treatment of neuropathic pain, and includes no mention of that condition or specific manifestations of neuropathy.

A number of patents discuss the use of individual compounds, sometimes found in essential oil mixtures, used to improve skin penetration. In U.S. Pat. No. 4,440,777, Zupan teaches the use of eucalyptol for its effect on enhancing the skin permeation of cosmetic and therapeutic agents. Further, in U.S. Pat. No. 4,931,283, Tusik teaches the use of menthol for the enhancement of transdermal drug delivery across mammalian skin. Specifically Zupan teaches enhancement of the delivery of non-steroidal anti-inflammatory agents selected from the group consisting of indomethacin, naproxen, fenoprofen, ibuprofen, sulindac and desoxysulindac. Tusik teaches enhancement of the delivery of the drugs propranolol, conjugated estrogens, etodalac, and 17β-estradiol.

A number of references also refer to the use of topical medications for the treatment of neuropathic pain. U.S. Pat. No. 6,638,981 describes an oil-in-water emulsion comprising an antidepressant, an NMDA-receptor antagonists, a lipophilic component, water; and a surfactant, which is applied topically to the skin for the treatment of neuropathic pain. U.S. Pat. No. 5,976,547 teaches the use of a combination of an herbal extract (arnica montana) with menthol crystal, camphor, oil of mint, eucalyptus oil, guaifenesin, non-steroidal anti-inflammatory medications, topical analgesics, or transdermal opioid analgesics. U.S. Pat. No. 5,260,313 presents a method of diagnosing and treating neuropathic pain syndromes with a composition of the essential oil extract of pelaroonium graveolens Ait.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide a homeopathic composition and method for the treatment of neuropathic pain. In accordance with an aspect of the present invention, there is provided a method for the treatment of neuropathic pain comprising topical administration of a composition comprising one or more homeopathic active ingredients combined with a base of one or more physiologically acceptable ingredients that enhances penetration of the homeopathic drug through the skin.

In accordance with another aspect of the invention, there is provided a composition comprising one or more homeopathic active ingredients combined with a base of one or more physiologically acceptable ingredients that enhances penetration of the homeopathic drug through the skin.

The current invention differs from known compositions and methods in a number of ways. Specifically, the use of homeopathic medication in a pharmaceutical product containing essential oils has been previously untested, in part, since traditional homeopathic practitioners consider essential oils to be contraindicated for concurrent use with homeopathic medications. Further, traditional homeopathies are prepared in an alcohol/water base which is not soluble in essential oil mixtures and not useful for effective topical administration of the homeopathic agents. Finally, the present inventors have now surprisingly found the use of homeopathic medications in combination with essential oils is effective for the treatment of neuropathic pain.

### BRIEF DESCRIPTION OF THE FIGURES

FIG. 1 graphically depicts the effect he topical application on pain reduction following the procedure of Example 1.

FIG. 2 graphically depicts the effect of a composition according to one embodiment of the invention (A) in comparison to a placebo (B).

### DETAILED DESCRIPTION OF THE INVENTION

Unless defined otherwise, all technical and scientific terms used herein have the same meaning as commonly understood by one of ordinary skill in the art to which this invention belongs.

Unless the context clearly indicates otherwise, as used herein plural forms of the terms herein are to be construed as including the singular form and vice versa.

The term “comprising” as used herein will be understood to mean that the list following is non-exhaustive and may or may not include any other additional suitable items, for example one or more further feature(s), component(s) and/or ingredients(s) as appropriate.

The present invention provides a solution to a number of unique and previously unsolved problems. Primarily, as mentioned in the background information, it is generally recognized that there is a limited ability to manage the symptoms of neuropathic pain. Current pharmaceutical treatments are often ineffective or result in unacceptable side effects at the doses necessary for symptom relief. The only approved over-the-counter drug, capsaicin, results in increased pain and burning at the site of application and is therefore not widely tolerated.

The traditional form of medicine known as “homeopathic medicine” utilizes a unique materia medica based on compounds found in the natural world. Homeopathic medications are prepared, by one skilled in the art, using the traditional techniques of dilution and succession, to various potencies. Although traditional textbooks of homeopathic medicine make no mention of “neuropathic pain” per se, homeopathic remedies can be chosen based on the unique symptoms of the neuropathic pain condition.

Traditional homeopathic remedies are prepared in an alcohol and water base and administered orally. The current invention is unique in that, for the purpose of relieving neuropathic pain, the formula which is the subject of the invention is applied topically to the area of pain and contains skin permeation enhancers.

The current invention utilizes traditionally prepared homeopathic medicines, but in a hydrophobic base comprising essential oils as skin permeation enhancers. To facilitate the solubility of the homeopathic active ingredients in a hydrophobic environment, the final dilution of the homeopathic medicines takes place in an alcohol environment (80-100% alcohol by volume). The exclusion of water enables the resultant homeopathic formulation to be soluble in an essential oil mixture or any individual essential oil compounds. This type of formulation is unique since those skilled in the art of homeopathy consider essential oils to be contraindicated for use combined with, or even concurrently with, homeopathics. For the treatment of neuropathic pain, however, it is desirable and efficacious to ensure the delivery of the homeopathics to the site of the aberrant nerve signal. This is accomplished with the addition of the essential oil components to the homeopathic(s), which facilitates transdermal delivery.

The term “essential oil”, as used herein, refers to a concentrated, hydrophobic liquid containing volatile aroma plant compounds isolated from a plant or derived synthetically. The essential oil comprises a variety of hydrophobic constituents (e.g., various terpenes, alcohol esters, aldehydes, ketones, phenols etc., typically soluble in water less than 10 wt %).

In most cases, essential oils are prepared by steam distillation, maceration, expression, and/or solvent extraction of plant materials, for example leaves and/or petals. Synthetic oil blends can also be utilized. Individual synthetic compounds, or natural compounds purified to homogeneity (e.g., synthetic or isolated terpenes, terpenoids, alcohol esters, ketones, phenols etc.) may also be utilized in the composition of the present invention. Similarly, one or more essential oils may be supplemented with one or more of the volatile compounds commonly found in plants or prepared synthetically.

In accordance with a specific embodiment of the present invention, the essential oil is from pelargonium, melaleuca (tea tree), bergamot, eucalyptus, lavender or a combination thereof.

All known essential oils are contemplated suitable for use herein. Suitable essential oils are prepared from plant material of one or more plant species using isolation methods well know to those skilled in the art, or prepared synthetically by one skilled in the art.

Essential oils are highly complex mixtures of often hundreds of individual compounds. A typical plant essential oil chromatogram may contain in the order of 200 or more distinct peaks. Plant essential oils are a complex mixture of terpenes, sesquiterpenes, esters, alcohols, phenols, aldehydes, ketones, organic acids, and various miscellaneous molecular structures. Furthermore, each class of compound above contains many subclasses. For example, the terpene classification includes hemiterpenes, monoterpenes, diterpenes, sesquiterpenes, triterpenes, tetraterpenes, and associated terpenoids formed by the modification or oxidation of the carbon skeleton. Essential oils often have an odour and are therefore used in food flavouring and perfumery. Essential oils are typically distinguished as a group by their minimal solubility in water, and this criteria makes them suitable for use in this invention.

Some examples of essential oils that would be suitable for use in this invention are; Agar (Aquilaria malaccensis), Ajwain (Carum copticum), Angelica root oil (Angelica archangelica), Anise (Pimpinella anisum), Asafoetida, Balsam (Myroxylon pereirae), Basil Bergamot, Black Pepper (Piper nigrum), Buchu, Cannabis, Caraway, Cardamom, Carrot seed, Cedarwood, Chamomile, Calamus, Cinnamon, Cistus species, Citronella, Clary Sage, Coffee, Coriander, Costmary, Costus, Cranberry, Cubeb, Cumin, Cypress, Cypriol, Curry Leaf, (Artemisia pallens), Dill, Elecampane, Eucalyptus, Fennell, Fenugreek, Fir, Frankincense, Galbanum, Geranium (and/or Pelargonium), Ginger, Go)denrod, Grapefruit, Henna, Helichrysum, Hyssop, Idaho Tansy, Jasmine, Juniper, Lavender, Laurus nobilis, Ledum, Lemongrass, Litsea cubeba, Marjoram, Melaleuca (Tea tree), Melissa (Lemon balm), Mentha arvensis (Mint), Mountain Savory, Mugwort, Mustard, Myrrh, Myrtle, Neroli, Nutmeg, Orange, Lemon, Oregano, Orris, Palo Santo, Parsley, Patchouli, Perilla, Petitgrain, Ravensara, Red Cedar, Roman Chamomile, Rose, Rosehip, Rosemary, Rosewood, Sage, Star anise, Sandalwood, Sassafras, Savory, Schisandra, Spearmint, Spikenard, Spruce, Tangerine, Tarragon, Tea tree oil, Thyme, Tsuga, Turmeric, Valerian, Vetiver (khus oil), Wintergreen, Ylang-ylang, and Zedoary.

In one embodiment of the invention, the essential oils include pelargoniumoil, bergamot oil, eucalyptus oil, lavender oil, and/or melaleuca oil.

Homeopathics are prepared in the manner practiced by one skilled in the art according to the Homeopathic Pharmacopoeia of the United States (HPUS), Good Manufacturing Practice (GMP), and applicable Over the Counter (OTC) regulations. Homeopathics chosen can be any of those based on traditional rubrics (symptom lists) from traditional textbooks of homeopathic medicine by one skilled in the art.

Examples of homeopathics traditionally used to treat the symptoms of neuropathic pain include Hypericum perforatum, Aconitum napellus, Secale cornutum, Rhus toxicodendron, Lycopodium, Phosphorus, Sulphur, Pulsatilla, Arsenicum, Nux vomica, Thuja occidentalis, Causticum, Kali carbonicum, Sepia, Silica, Conium maculatum, Apis Mellifica, Belladonna, Agaricus, Platina, Calcarea Phosphorica, Chininum, Coccus cacti, Rhododendron chrysanthum OR Rhodium metallicum, Graphites, Colocynthis, Mercurius, Stannum metallicum, Phosphoricum acidum, Lachesis, Capsicum, Staphylococcinum, Natrum mur, Colchicinum, Bryonia, Ferrumphos, Allium cepa, Agrentum metallicum, Baryta-carb, Zincum, Chamomilla, Mezereum, Ranunculus bulbases, Ammonium muriaticum, Euphrasia, Sabadilla officinalis, Asafoetida, Secale comutum, Carbo-veg, Plumbum metallicum, Nit-ac, Spigelia, Carbo animalis, Cina, Kali nitricum, Chelidonium, Dulcamara, Aurum metallicum, Ledum, Sabina officinalis, Ignatia amara, Digitalis, Carbon sulphuratum, Hepar sulph, Kali-bich, Amonium carb, Cuprum metallicum, Magnesia phosphoric, Iodine, Veratinum, Guai, Calcphos, Mercurius corrosivus OR Mercurius cyanatus, Spongia tosta, Nux Moschata, Cantharis, Kreosotum, Taraxacum officinale, Anacardium orientale, Camphor, Oleander, Berberis, Manganum, Muriaticum acidum, Naturm Carb, Valeriana officinalis, Kali-sulph, Laurocerasus officinalis, Ambra Grisea, Asarum, Sulphuricum acidum, Ant crud, Cicuta, Mag-mur, Kali-phos, Clemetiserecta, and Kali arsenicum.

Six examples of such remedies are hypericum per oratum, phosphorus, rhus toxicodendron, sectile cornutum, lycopodium and aconitum napellus.

The potency of the homeopathic ingredients may vary from mother tincture (undiluted) to 1M, however the preferred embodiment recommends a homeopathic OTC potency of 3× to 30C, with 12C ideal. The homeopathic ingredients may be added to the essential oils in an amount varying from 0.1% by volume to 50% by volume, with 1% to 10% by volume preferred.

The composition described herein can be delivered to the skin overlying the area of aberrant nerve function or neuropathic pain in a number of ways. The composition can be applied topically directly to the area using the finger or other instrument of application. The composition can also be delivered via a container suitable for topical applications to the skin, such as a container with the ability to spray, roll or otherwise apply the composition to the skin. The composition can also be added to a suitable material containing a reservoir and adhesive for application to the skin.

In accordance with a particular embodiment of the present invention, the composition consists only of an essential oil or mixture of essential oils as a base and one or more homeopathic active agents. Alternatively, the composition additionally comprises a pharmaceutically acceptable diluent or excipient suitable for use in a topical composition. Selection of such additional diluents or excipients is within the abilities of a person of ordinary skill in the field and is made based on the intended use of the product. The additional diluents or excipients do not affect the therapeutic efficacy of the composition.

In accordance with another embodiment of the invention, the composition is or can be incorporated into a device containing a reservoir for the sustained release of medication to be absorbed topically through the skin. The medication within the reservoir migrates over time from within the reservoir to the site of action. The reservoir is supported by a backing structure and is attached to the skin via a suitable adhesive. Alternatively, the composition and adhesive are comingled in the reservoir. Treatment involves placing the device on the skin for a prescribed duration.

In accordance with one embodiment of the invention, the composition is prepared using a suitable gelling agent. For example, a combination of bees wax and sorbitan monopalmitate can be used as a gelling agent. Selection of a suitable gelling agent is made to ensure proper consistency and absorption without negatively affecting therapeutic activity or being an irritant.

The compositions of the present invention are useful in treating neuropathic pain. Neuropathic pain can be caused by a disorder selected from, but not limited to, diabetic peripheral neuropathy, herpes zoster, post herpetic neuralgia, trigeminal neuralgia, complex regional pain syndrome, reflex sympathetic dystrophy, phantom limb syndrome, neuropathic pain due to chronic disease (multiple sclerosis, HIV, etc), neuropathic pain due to trauma (causalgia), neuropathic pain due to impingement (ie. sciatica, carpal tunnel, etc), neuropathic pain due to drug exposure or toxic chemical exposure, neuropathic pain due to infection or post infection, neuropathic pain due to impaired organ function, neuropathic pain due to vascular disease, neuropathic pain due to metabolic disease, neuropathic pain due to cancer or cancer treatment, neuropathic pain due to autoimmune disease, neuropathic pain due to fibromylagia, and neuropathic pain with no known cause (idiopathic), or any in is that is characterized by burning sensations, shooting pain, numbness, tingling, allodynia or a combination thereof.

To gain a better understanding of the invention described herein, the following examples are set forth. It should be understood that these examples are for illustrative purposes only. Therefore, they should not limit the scope of this invention in any way.

### EXAMPLES

### Example 1

Community-dwelling individuals with diagnosed neuropathic pain were recruited from the community. No distinction was made with respect to the underlying cause of the neuropathic pain. Potential participants were excluded from the study if they presented with (1) an inability to walk at least 20 meters independently, (2) a history or evidence of central nervous system dysfunction, (3) musculoskeletal injury and or deformity that may influence gait and posture, (4) a history of vestibular dysfunction, (5) evidence of plantar cutaneous ulcer, and (6) any uncontrolled metabolic, cardiovascular, or respiratory disease. Following explanation of all the details of the study, each participant signed an informed consent. The project was approved by the Institutional Review Board.

Participants were randomly assigned into a treatment or no treatment group. The cause of the peripheral neuropathy included diabetes mellitus (n=6) and trauma (n=2), with the remaining cases of indeterminate cause (n=6). The treatment group received external application of a composition containing the homeopathic ingredient hypericum perforatum combined with an essential oil mixture of lavender, pelargonium, bergamot, eucalyptus and tea tree oil. Before each application pain level was monitored on a 0-10 visual scale. 15 subjects were recruited for each group, but only five (1man, 4 women , age=66±17 years, height=165±8 cm, body mass=79±25 kg) and nine (5 men, 4 women, age=64±15 years, height=177±11 cm, body mass=101±24 kg) completed all the required testing.

Pain level associated with the feet was self-monitored on a 0-10 scale (10 being most severe) three times every session. Participants recorded their pain level immediately upon arrival (PRE), five minutes following composition application in the treatment group (or five minutes of quiet sitting in no treatment group) (5 MIN), and immediately following one hour of exercise (POST). The pain relieving composition was applied with a spray top applicator for a total of five pumps per foot, with one pump over (1) the dorsal aspect of the toes, (2) the dorsal foot roughly half way between the toes and the ankle, (3) the inside of the foot midway along the longitudinal arch, (4) the outside of the foot midway between the toes and ankle, and (5) the plantar surface of the foot half way between the heel and toes. Total volume of composition applied was approximately 0.75 ml to each foot.

Pre- and post-test comparison within each group were statistically tested with one tailed T-Tests. Longitudinal pain level changes of the two groups were tested using trend analysis. The slopes of the regression lines were compared to a horizontal line for an absolute effect and with each other for group comparison. The relative post- to pre-test changes of each criterion measure were compared between groups with one tailed T-Tests. Significance was set at α=0.05.

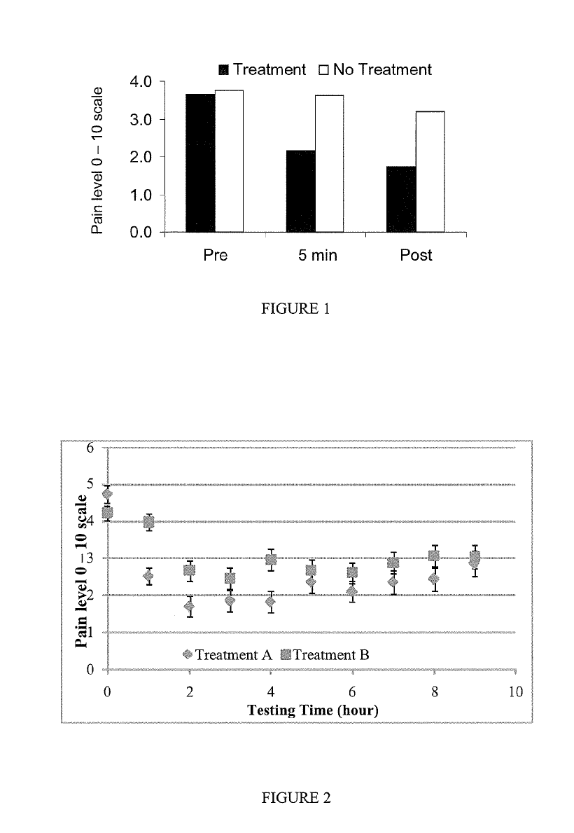

The effect of the topical application on pain reduction are presented in FIG. 1, in which pain scales were averaged across the six weeks of the study. Average PRE pain levels were 3.7 and 3.8 for the treatment and no treatment groups, respectively. Application of the topical homeopathic/essential oil composition significantly reduced the 5 MIN (2.2) and POST (1 hr) (1.7) pain levels.

### Example 2

A composition containing the homeopathic ingredient hypericum perforatum combined with an essential oil mixture of lavender, pelargonium, bergamot, eucalyptus and tea tree was tested in a double blind placebo controlled fashion. Sixty subjects with plantar cutaneous (foot sole) pain due to all cause neuropathy were recruited from the community. Each subject was assessed for inclusion/exclusion based on standard criteria for neuropathic pain studies. Subjects found suitable were given the opportunity to participate once they signed a consent form. Each subject was also be provided with an adverse events report form, and instructed in its use.

Change in average pain levels (10 point numeric scale) at 30 min, 1 hr, 2 hr, 3 hr, 4 hr, 5 hr, 6 hr, 7 hr, and 8 hr after application of the pain relieving homeopathic/essential oil composition was recorded. Change in pain as reported on the McGill Pain Questionnaire (short form) 30 min after application was also measured. Each participant received either the active treatment or mineral oil placebo in random order, based on a balanced latin square design. The identities of the supplied bottled sprays were not known to the investigators or the participants. Each treatment was repeated at least once in each participant to test the reliability of the testing. The 10 point numerical pain scale was programmed in a pocket PC and presented to the participants automatically on the PC's screen accompanied with an audio reminder.

The pain relieving composition was applied with a spray top applicator for a total of five pumps per foot, with one pump over (1) the dorsal aspect of the toes, (2) the dorsal foot roughly half way between the toes and the ankle, (3) the inside of the foot midway along the longitudinal arch, (4) the outside of the foot midway between the toes and ankle, and (5) the plantar surface of the foot half way between the heel and toes. Total volume of composition applied was approximately 0.75 ml to each foot.

Treatment with the homeopathic/essential oil composition resulted in a statistically significant reduction in spontaneous pain (p<0.005) which was in effect within 30 minutes and lasted approximately 8 hrs (FIG. 2).

### Example 3

M.D. a 56 yr old woman with a 34 yr history of DM Type 1 reported to the clinic. Her complaints included angina, poor vision, swollen feet, chronic pain in feet as well as numbness and burning sensations. Her weight was 249 lb and her height 5′5″.

Blood work was as follows:
Fasting Glucose 8.3 (3.6-5.6)
HA1C 9.2% (4.6-6.5)
Creatinine 194 (35-88)
Urea 11.6 (2.9-9.3)
Liver enzymes normal

Her only medication was insulin for diabetes. She was assessed for pain using a 0-10 digital scale with the yardsticks 0=no pain and 10=worst possible pain. She rated her pain level as 8/10 in both feet. Her left foot was then treated with a thin film of a cream consisting of 1% homeopathic ingredients (equal parts Hypericum perforatum, Aconitum napellus, Secale cornutum, Rhus toxicodendron, Lycopodium, and Phosphorus all at 12C potency) prepared in a non-medicinal cream base consisting of water, glycerin, distearyldimonium chloride, petrolatum, isopropyl palmitate, cetyl alcohol, benzoyl alcohol and sodium chloride). Her right foot was treated with a thin film of the same 1% homeopathic ingredients (Hypericum perforatum, Aconitum napellus, Secale cornutum, Rhus toxicodendron, Lycopodium, and Phosphorus all at 12C potency) prepared in a base consisting of an essential oil mixture of 28.29% v/v lavender, 28.29% v/v pelargonium graveolens,14.14% v/v citrus bergamia, 14.14% v/v eucalyptus globulus and 14.14% v/v melaleuca alternifolia. After 5 minutes of quiet sitting she reported a pain level of 6/10 in the left foot and 2/10 in the right foot. After one hour she reported that her left foot was still painful and she asked to use the essential oil formula on the left foot. She proceeded to do so and received the same degree of pain relief as experienced in the right foot.

Table 1 provides a list of the components within the essential oil blend used as the base in this Example.

TABLE 1

| Compound | |
|---|---|
| cis-3-hexen-1-ol | |
| <-thujene | |
| <-pinene | |
| camphene | |
| sabinene | |
| <-pinene | |
| myrcene | hexyl butyrate |
| <-phellandrene | nerol |
| <-terpinene | citronellol |
| p-cymene | neral |
| limonene | cis-iso-geraniol |
| 1,8-cineole | geraniol |
| benzyl alcohol | linalyl acetate |
| cis-<-ocimene | geranial |
| trans-<-ocimene | citronellyl formate |
| <-terpinene | neryl formate |
| trans-linalool oxide (fur.) | lavandulyl acetate |
| terpinolene | geranyl formate |
| cis-linalool oxide (fur.) | citronellyl acetate |
| linalool | neryl acetate |
| cis-rose oxide | <-copaene |
| phenylethyl alcohol | <-bourbonene |
| trans-rose oxide | geranyl acetate |
| dihydrolinalool | longifolene |
| camphor | <-caryophyllene |
| menthone | decyl acetate |
| isoborneol | trans<□αbergamotene |
| isomenthone | <-guaiene |
| borneol | 6,9-guaiadiene |
| lavandulol | muurola-3,5-diene |
| terpin-1-en-4-ol | citronellyl propionate |
| isomenthol | <-humulene |
| <-terpineol | alloaromadendrene |
| <-terpineol | geranyl propionate |

### Example 4

R.M. a 58 year old male with post shingles pain (post herpetic neuralgia) of the left lateral trunk region of 6 months duration. He reported almost constant pain, with pricking and burning sensations, worse from touch. He was taking 20 mg Lipitor per day for cholesterol management and no other medications. He was treated with a thin film of a cream consisting of 1% homeopathic ingredients (equal parts Hypericum perforatum, Aconitum napellus, Secale cornutum, Rhus toxicodendron, Lycopodium, and Phosphorus all at 12C potency) prepared in a non-medicinal cream base consisting of water, glycerin, distearyldimonium chloride, petrolatum, isopropyl palmitate, cetyl alcohol, benzoyl alcohol and sodium chloride). He reported a reduction in pain from 7/10 pre treatment to 4/10 five minutes post treatment. On a subsequent clinic visit he was treated with a thin film of the same 1% homeopathic ingredients (Hypericum perforatum, Aconitum napellus, Secale cornutum, Rhus toxicodendron, Lycopodium, and Phosphorus all at 12C potency) prepared in a base consisting of a synthetic essential oil blend (Table 2). After 5 minutes of sitting he reported a pain level of 1/10, the most relief he had experienced from any treatment tried since the onset of his pain.

TABLE 2

| | |
|---|---|
| cis-hex-3-en-1-ol | trans-iso-geraniol |
| linalool | neral |
| phenylethyl alcohol | cis-iso-geraniol |
| cis rose oxide | geraniol |
| trans rose oxide | linalyl acetate |
| menthone | geranial |
| isoborneol | citronellyl formate |
| isomenthone | neryl formate |
| borneol | geranyl formate |
| P-terpineol | furanopelargone A |
| nerol | geranyl tiglate |
| citronellol | |

### REFERENCES

1. Kalso, E Sodium Channel Blockers in Neuropathic Pain, Current Pharmaceutical Design, Volume II, Number 23, September 2005, pp. 3005-3011(7)
2. Sindrup, Søren H.; Otto, Marit; Finnerup, Nanna B. Jensen, Troels S. Antidepressants in the Treatment of Neuropathic Pain Basic & Clinical Pharmacology & Toxicology, Volume 96, Number 6, June 2005, pp. 399-409(11)
3. Jensen T S. Anticonvulsants in neuropathic pain: rationale and clinical evidence. Eur Pain. 2002; 6 Suppl A:61-8
4. Kingery W S. A critical review of controlled clinical trials for peripheral neuropathic pain and complex regional pain syndromes. Pain 1997; 73:123-39.
5. Elon Eisenberg, MD; Ewan D. McNicol, RPh; Daniel B. Carr, MD Efficacy and Safety of Opioid Agonists in the Treatment of Neuropathic Pain of Nonmalignant Origin JAMA. 2005; 293:3043-3052.
6. Abdullah D, Ping Q N, Liu G J T I. Enhancing effect of essential oils on the penetration of 5-fluorouracil through rat skin. Yao Hsueh Hsueh Pao . 1996; 31(3):214-221.

| | | |
|---|---|---|
| U.S. Pat. No. 7,229,648 | June 2007 | Dreyer |
| U.S. Pat. No. 6,132,760 | October 2000 | Hedenstrom et al. |
| US ,579,543 | June 2003 | McClung. |
| U.S. Pat. No. 4,440,777 | April 1984 | Zupan |
| U.S. Pat. No. 4,931,283 | June 1990 | Tsuk |
| U.S. Pat. No. 6,638,981 | October 2003 | Williams et al. |
| U.S. Pat. No. 5,976,547 | November 1999 | Archer et al |
| U.S. Pat. No. 5,260,313 | November 2003 | Frome |
| US20060275509 | December 2006 | Wegener |
| US20030161867 | August 2003 | Lu et al. |

All publications, patents and patent applications mentioned in this Specification are indicative of the level of skill of those skilled in the art to which this invention pertains and are herein incorporated by reference to the same extent as if each individual publication, patent, or patent applications was specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

1.-9. (canceled)

10. A composition comprising one or more homeopathic active ingredients combined with a base comprising one or more physiologically acceptable plant essential oils.

11. The composition of claim 10, wherein one of the one or more homeopathic active ingredients is Hypericum perforatum, Aconitum napellus, Secale cornutum, Rhus toxicodendron, Lycopodium, or Phosphorus.

12. The composition of claim 11, wherein the homeopathic active ingredient has a potency ranging from tincture to about 1M, or from about 3× to about 30C, or about 12C.

13. The composition of claim 10, wherein the composition comprises a combination of Hypericum perforatum, Aconitum napellus, Secale cornutum, Rhus toxicodendron, Lycopodium, and Phosphorus.

14. The composition of claim 10, wherein the plant essential oils are selected from the group consisting of pelargonium, melaleuca, bergamot, eucalyptus, lavender and combinations thereof.

15. The composition of claim 10, wherein the homeopathic active ingredients are added to the base in an amount equal to approximately 0.1 to 50% by volume.

16. The composition of claim 10, wherein the base is a gel base manufactured with a suitable gelling agent.

17. The composition of claim 10, which is a spray, an ointment, or a roll-on or is formulated for administration via a skin patch.

18. (canceled)

19. A method for the treatment of neuropathic pain comprising topical administration of a composition comprising one or more homeopathic active ingredients combined with a base of one or more physiologically acceptable plant essential oils.

20. The method of claim 19, wherein one of the one or more homeopathic active ingredients is Hypericum perforatum, Aconitum napellus, Secale cornutum, Rhus toxicodendron, Lycopodium, or Phosphorus.

21. The method according to claim 20, wherein each homeopathic active ingredient has a potency ranging from tincture to about 1M, or from about 3× to about 30C, or about 12C.

22. The method of claim 19, wherein the composition comprises a combination of Hypericum perforatum, Aconitum napellus, Secale cornutum, Rhus toxicodendron, Lycopodium, and Phosphorusone or more physiologically acceptable ingredients is one or more plant essential oils.

23. The method of claim 22, wherein the plant essential oils are selected from the group consisting of pelargonium, melaleuca, bergamot, eucalyptus, lavender and combinations thereof.

24. The method of claim 19, wherein the homeopathic active ingredients are added to the base in an amount equal to approximately 0.1 to 50% by volume.

25. The method of claim 19, wherein the neuropathic pain is caused by a disorder selected from the group consisting of diabetic peripheral neuropathy, herpes zoster, post herpetic neuralgia, trigeminal neuralgia, complex regional pain syndrome, reflex sympathetic dystrophy, phantom limb syndrome, neuropathic pain due to chronic disease, neuropathic pain due to trauma, neuropathic pain due to impingement, neuropathic pain due to drug exposure or toxic chemical exposure, neuropathic pain due to infection or post infection, neuropathic pain due to impaired organ function, neuropathic pain due to vascular disease, neuropathic pain due to metabolic disease, neuropathic pain due to cancer or cancer treatment, neuropathic pain due to autoimmune disease, neuropathic pain due to fibromylagia, and neuropathic pain with no known cause and any pain is that is characterized by burning sensations, shooting pain, numbness, tingling, allodynia or a combination thereof.

26. The method of claim 19, wherein the base is a gel base manufactured with a suitable gelling agent.

27. The method of claim 19, wherein the delivery method is via a spray, ointment, device that adheres to the skin, or roll-on.
